(19) United States
(12) Patent Application Publication
(10) Pub. No.: US 2002/0187957 A1
(43) Pub. Date: Dec. 12, 2002

(54) Time release reverse transcriptase inhibitors

(76) Inventor: Bruce Halstead, Grand Terrace, CA (US)

Correspondence Address: Robert D. Fish, Suite 1400, 611 Anton Blvd., Costa Mesa, CA, 92626, US

(21) Appl. No.: 10/159,417

(22) Filed: May 29, 2002

Related U.S. Application Data
(60) Provisional application No. 60/294,477, filed on May 30, 2001.

(51) Int. Cl. A61K 31/7072; A61K 31/655; A61K 35/78

(52) U.S. Cl. 514/50; 514/151; 424/725

(57) ABSTRACT

A pharmacological composition comprises a reverse transcriptase inhibitor in a quantity sufficient to reduce a viral serum titer of a virus in an amount of at least 20% over a period of at least 6 hours, wherein preferred reverse transcriptase inhibitor comprise a plant extract.

This application claims the benefit of U.S. provisional application No. 60/294477 filed May 30, 2001, incorporated herein by reference in its entirety.

### FIELD OF THE INVENTION

The field of the invention is antiviral compositions.

### BACKGROUND OF THE INVENTION

Numerous antiviral drugs are known in the art, however, all or almost all of them suffer from one or more disadvantages. Particularly problematic in the administration is of such drugs is their relatively low solubility and/or comparably short serum half-life time. Consequently, many patients need to follow a strict regimen to maintain effective serum concentration of such drugs, frequently resulting in repeated disruptions of an otherwise productive lifestyle. Therefore, there is a need for improved antiviral compositions that are well tolerated, simple to administer, and maintain a relatively long serum half-life.

### DETAILED DESCRIPTION

The inventors contemplate that treatment of a viral infection can be significantly improved by administration of an antiviral agent in a time-release formulation. More specifically, the inventors contemplate that a reverse transcriptase inhibitor in a time-release formulation is administered to a patient suffering from a viral infection. Particularly contemplated viruses include retroviruses (e.g., HIV, HCV), ssDNA and dsDNA.

In an especially preferred aspect, the reverse transcriptase inhibitor (RTI) is an extract from a plant that is known to have an antiviral effect, or an isolated or synthetically prepared compound that can be found in a plant known to have an antiviral effect. Especially contemplated plants include Abies webbiana; Acacia spec. Acacia Arabia; Agrimonia eupatoria; Ajuga decumbens; Allium cepa; Allium sativum; Aloe vera; Altemanthera philoxeroides or sessiles; Ammi maius; Andographis paniculata; Apium graveolens; Apium leptophyllum; Arachis hypogaea; Arctium lappa; Amebia euhcroma; Asparagus racemosus; Astragalus spinosus; Astragalus lentingosis swainsonine; Buchenavia capita; Bryonia cretica ssp. Dioica; Bryonia angustifolia; Camellia theifera; Camellia sinensis; Cedrela toona; Chrysanthemum morifolium; Coffea arabica; Coptis chinesis; Coptis teetoides; Coptis japonica; Coraria nepalensis; Coriandrum sativum; Curcuma longa; Datura metel syn alba; Daucus carota; Echinacea angustiflora and purpurea; Echinacea simulata; Echinacea pallida; Epimedium grandiflorum; Epimedium sagittatum; Epimedium sinense; Epilobium angustifolium; Erigeron Canadensis; Eugenia or Syzigium claviflorum; Fagara xanthox; Foeniculum vulgarel; Gardenia coronaria; Gaultheria trichophylla; Glycine max; Glycyrrhiza labra; Gossypium herbaceum; Heracleum sphondylium; Hypericum perforatum; Hypericum japonicum; Hyssopus officinalis; Jasminum officinale; Lithospermum erythrorhizon; Lonicerajaponica; Luffa luffa; Lycopus europaeus; Magnolia officinalis; Mallotus repandus; Mallotus philippinesis; Matricaria chamomil; Matricaria recutitia; Melissa parviflora; Melissa officinalis; Momordica balsamina; Momordica charantia; Narcissus tazetta; Narcissus pseudonarcissus; Oenthera rosea; Paeonia spec.; Papaver somniferum; Perilla frutescens; Phyllanthus niruri; Pinus koraicenis; Pinus parviflora; Piper nirgum; Plumeria rubra; Polyantha suberosa; Prunella vulgaris; Prunus bakariensis; Prunus amygdalus; Psoralea corylifolia; Randia dunatorum; Raphanus sativus; Rheum palmatum; Rhus coriaria; Rhus chinesis; Ricinus communis; Rosmarinus officinalis; Salvia miltiorhiza and officinalis; Sambucus ebulus; Saussurea lappa; Scilla griffithii; Scutellaria baicalensis baiealein; Sedum sediforme; Senecio scandens; Senecio aereus; Skimmia laureola; Solarium niporum; Swertia franchetiana; Terminalia chebula; Terminalia catappa; Terminalia alata; Thula occidentalis; Trapalaponica spec.; Trichosanthes dioica; Trichosanthes kirilowii; Urtica dioica; Viola yeodensis; Woodfordia fruticosa; Woodwardia spec. Zanoxylum nitidum.

Alternatively it should be appreciated that RTIs other than plant extracts are also appropriate, and such agents particularly include known and commercially available RTIs as indicated in

TABLE 1

| Drug | Generic Name | Brand Name | Analogue |
|---|---|---|---|
| 3TC | lamivudine | Epivir/3TC | cytidine |
| ABC | abacavir | Ziagen | guanosine |
| AZT | zidovudine | Retrovir | thymidine |
| ddC | zalcitabine | HIVID | cytidine |
| ddI | didanosine | Videx | adenosine |
| d4T | stavudine | Zerit | thymidine |
| F-ddA | lodenosine | | adenosine |
| FTC | emtricitabine | Coviracil | cytidine |
| PMEA | adefovir dipivoxil | Preveon | adenosine |
| PMPA | tenofovir disoproxil | adenosine | |

In further especially preferred aspects, contemplated antiviral agents may include a chelating agent that chelates a bivalent metal ion, preferably Mg²⁺ and/or Ca²⁺. Especially preferred chelating agents include EDTA, EGTA, 1,2-Bis(2-aminophenoxy)ethane-N,N,N′,N′-tetraacetic acid, Ethylenebis(oxyethylenenitrilo)tetraacetic acid, 1,2-bis(2-aminophenoxy)ethane-N,N,N′,N′-tetraacetic acid tetrakis(acetoxymethyl ester), trans-1,2-diaminocyclohexane-tetraacetic acid, and diethyllenetriamine-pentaacetic acid, trimethylaminetricarboxylic acid, poly(aspartic acid), and poly(glutamic acid).

There are numerous known methods of preparing a time-release formulation, and all of the known methods are contemplated suitable for use in conjunction with the teachings herein. Particularly contemplated time release formulations include ion exchange resins, encapsulations with acid or base resistant coatings, compacting the formulation to control solvation, slow-melting carriers, enzyme-degradable carriers, etc.

With respect to the dosage of contemplated compositions, it is contemplated that the RTI is present in a single dose in a concentration such that the viral titer is reduced at least 20% over a period of at least 6 hours, more at least 30% over a period of at least 8 hours, and most preferably at least 45% over a period of at least 12 hours. Furthermore, it is contemplated that where contemplated compositions further comprise a chelating agent, the chelating agent is present in a single dose in a concentration such that the serum Mg²⁺ and/or Ca²⁺ concentration is reduced at least 20% over a period of at least 6 hours, more preferably at least 35% over a period of at least 12 hours, and most preferably at least 45% over a period of at least 12 hours.

With respect to the administration of contemplated compositions, it should be recognized that various protocols are suitable, and especially contemplated protocols include oral, topical, and parenteral administration. Consequently, the formulation of contemplated compositions may vary substantially, however, it is preferred that the RTI is administered in approved and/or known formulations.

Thus, specific embodiments and applications of time release RTIs have been disclosed. It should be apparent, however, to those skilled in the art that many more modifications besides those already described are possible without departing from the inventive concepts herein. The inventive subject matter, therefore, is not to be restricted except in the spirit of the appended contemplated claims. Moreover, in interpreting both the specification and the contemplated claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

What is claimed is:

1. A pharmacological composition, comprising:
an reverse transcriptase inhibitor in a quantity sufficient to reduce a viral serum titer of a virus in an amount of at least 20% over a period of at least 6 hours.

2. The composition of claim 1 wherein the reverse transcriptase inhibitor is present in a quantity sufficient to reduce the viral serum titer of the virus in an amount of at least 30% over a period of at least 8 hours.

3. The composition of claim 1 wherein the reverse transcriptase inhibitor is present in a quantity sufficient to reduce the viral serum titer of the virus in an amount of at least 45% over a period of at least 12 hours.

4. The composition of claim 1 wherein the reverse transcriptase inhibitor comprises a plant extract.

5. The composition of claim 1 wherein the reverse transcriptase inhibitor comprises a compound that is found in a plant demonstrated to have an antiviral effect.

6. The composition of claim 1 wherein the reverse transcriptase inhibitor is synthesized de novo.

7. The composition of claim 1 wherein the reverse transcriptase inhibitor is selected for the group consisting of lamivudine, abacavir, zidovudine, zalcitabine, didanosine, stavudine, lodenosine, emtricitabine, adefovir dipivoxil, and tenofovir disoproxil.

8. The composition of claim 1 further comprising a chelator.

9. The composition of claim 8 wherein the chelator chelates at least one of Ca²⁺ and Mg²⁺.

10. The composition of claim 8 wherein the chelator is selected from the group consisting of 1,2-Bis(2-aminophenoxy)ethane-N,N,N′,N′-tetraacetic acid, Ethylenebis(oxyethylenenitrilo)tetraacetic acid, 1,2-bis(2-aminophenoxy)ethane-N,N,N′,N′-tetraacetic acid tetrakis(acetoxymethyl ester), trans-1,2-diaminocyclohexane-tetraacetic acid, and diethyllenetriamine-pentaacetic acid.

11. The composition of claim 8 wherein the chelator is selected from the group consisting of trimethylaminetricarboxylic acid, poly(aspartic acid), and poly(glutamic acid).

12. The composition of claim 8 wherein the chelator is ethylenediamine-N,N,N′,N′-tetraacetic acid.

13. The composition of claim 13 wherein the virus is a retrovirus.

14. The composition of claim 14 wherein the retrovirus is an HIV virus or an HCV virus.
